(19) United States
(12) Patent Application Publication
(10) Pub. No.: US 2016/0220479 A1
(43) Pub. Date: Aug. 4, 2016

(54) TOPICAL COMPOUNDS CONTAINING ADIPOSE-DERIVED HORMONES FOR THE REJUVENATION OF SKIN

(71) Applicant: OHIO STATE INNOVATION FOUNDATION, Columbus, OH (US)

(72) Inventors: Matthew DURING, Weston, CT (US); Ergun KOCAK, Columbus, OH (US)

(73) Assignee: OHIO STATE INNOVATION FOUNDATION, Columbus, OH (US)

(21) Appl. No.: 15/021,455

(22) PCT Filed: Sep. 12, 2014
(86) PCT No.: PCT/US2014/055481

§ 371 Date: Mar. 11, 2016

Related U.S. Application Data
(60) Provisional application No. 61/877,018, filed on Sep. 12, 2013.

(51) Int. Cl. A61K 8/98; A61K 8/02; A61K 8/92; A61K 8/49; A61Q 19/08; A61K 8/31

(52) U.S. Cl. CPC A61K 8/981; A61Q 19/08; A61K 8/31; A61K 8/92; A61K 8/925; A61K 8/49; A61K 8/0204; A61K 2800/82

(57) ABSTRACT

Disclosed are cosmetic compositions comprising lysed adipose tissue. The lysed adipose tissue contains no live cells. The cosmetic compositions can be formulated into topical formulations. Also disclosed are methods of making and using the cosmetic compositions comprising lysed adipose tissue. The cosmetic compositions can be made by obtaining fat tissue from a subject, lysing the fat tissue and combining the lysed fat tissue with a topical base. The cosmetic compositions can be used to rejuvenate or enhance skin.

### CROSS REFERENCE TO RELATED APPLICATIONS

This application claims the benefit of priority to U.S. Provisional Application 61/877,018, filed Sep. 12, 2013, which is incorporated by reference herein in its entirety.

### FIELD

This disclosure relates to compositions comprising lysed adipose tissue. These compositions can be used to rejuvenate the skin and provide a more youthful appearance.

### BACKGROUND

The skin is the body's first line of defense. Skin protects the body from environmental factors and physical damage. The skin can be damaged by a wide array of factors such as but not limited to the sun, extreme temperatures, malnutrition, stress, fatigue and microbes. The skin is also affected by the general aging process. All of these factors can change the appearance, physical properties or physiological functions of the skin in ways that would be considered undesirable. Changes in the skin include but are not limited to wrinkles, reduced elasticity, reduced firmness, dryness, and sun spots or other discolorations.

There are several topical formulations being studied that either contain live cells, such as adipocyte stem cells or purified leptin. The use of live cells in a formulation can lead to excess scrutiny and fears due to the fact that a live biologic is present in the formulation.

Purified leptins have proven effective but it can be expensive to purify or synthesize leptin. Furthermore, leptin may not be the only protein present in the live cells that can provide beneficial effects to the skin. For example, other adipokines that are endogenously present in adipose tissue, such as but not limited to adiponectin, visfatin and apelin, may be able to contribute to improving or restoring the skin. Therefore, a topical formulation that does not contain live cells and does not require a purified or synthetic leptin would be beneficial. Thus, there is a need to identify new compositions and methods for managing and improving skin health and maintaining normal skin appearance and restoring aged skin to a youthful appearance.

### SUMMARY

Compositions and methods for rejuvenating skin are described. Rejuvenating the skin can result in fewer wrinkles or fuller skin. The composition for rejuvenating skin can comprise lysed adipose tissue and a dermatologically acceptable vehicle. The lysed adipose tissue can comprise lysed adipocytes, preadipocytes, fibroblasts, endothelial cells, multipotent stem cells, and combinations thereof. In some examples, the lysed adipose tissue can comprise intracellular, extracellular, and interstitial contents of adipocytes. The intracellular components can include adipokines and/or adipose-derived hormones.

The adipose tissue can be obtained from any suitable source. In some examples, the adipose tissue can be obtained from a subject. The adipose tissue can be autologous to the subject being administered the cosmetic composition. In some aspects, the adipose tissue can be obtained from a subject other than the subject being administered the cosmetic composition. Thus, the adipose tissue can be autologous, allogeneic or xenogeneic to the subject to whom the cosmetic composition comprising the lysed adipose tissue is being administered to.

The cosmetic compositions can comprise a dermatologically acceptable vehicle. The dermatologically acceptable vehicle can comprise water, mineral oil, petrolatum, ceresin, lanolin alcohol, methylchloroisothiazolinone, and methylisothiazolinone. The cosmetic compositions can also comprise a moisturizing agent or a humectant.

The cosmetic compositions can be in the form of a lotion, cream, gel, serum, or emulsion. In some examples, the composition can be a cleanser, a toner, a moisturizer, or a mask. The cosmetic compositions can comprise about 0.001% to about 80% by weight of lysed adipose tissue. In some aspects, the composition comprises about 2% to about 5% by weight of lysed adipose tissue. The lysed adipose tissue can be at a concentration of from about 50 μg/mL to about 1 mg/mL of the composition.

Methods of preparing a fat lysate topical formulation are also provided. The method can comprise a) removing adipose-containing tissue from a subject; b) lysing the adipose-containing tissue; and c) combining the lysed adipose-containing tissue with a topically applicable base. The method can also comprise filtrating or purifying the lysed adipose-containing tissue before combining with the topically applicable base. In some examples, lysing the adipose-containing tissue can eliminate any live cells.

The adipose tissue can be removed by a lipectomy procedure. The lipectomy procedure can be suction assisted lipectomy (liposuction), dermal lipectomy procedures used for body contouring (such as abdominoplasty, brachioplasty, thigh-plasty, belt-lipectomy, buttock lifting), and isolated lipectomy (direct excision or suction assisted) to obtain fat for the cream only. In some examples, the adipose tissue can be subcutaneous fat. The subcutaneous fat can be from any part of the body. In some examples, the adipose tissue is not fat taken from the breast or other non-subcutaneous sources (such as intra-abdominal).

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the compositions and methods as claimed.

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By “adipose tissue” or “fat” is meant connective tissue composed mostly of adipocytes. The adipose tissue can be white or brown and subcutaneous or visceral, or any combination thereof.

“Lysed adipose tissue” or “fat lysate” can be used interchangeably and refers to adipose tissue that has been treated with one of many known lysing techniques. Lysed adipose tissue does not contain any living cells. Lysed adipose tissue can be composed of cellular, intracellular, extracellular, and interstitial contents of both lysed adipocytes and all of the other lysed cells found in the tissue, including vasculature and connective tissue. The term “lysed adipose tissue” can also refer to individual or a combination of cells or cellular components present in adipose tissue. For example, lysed adipose tissue can refer to lysed adipocytes, preadipocytes, fibroblasts, endothelial cells, multipotent stem cells, adipokines, adipose-derived hormones, or combinations thereof.

“Adipose-containing tissue” refers to any tissue that contains adipose or fat. Adipose-containing tissue includes adipose tissue. Adipose-containing tissue includes the tissue obtained from any procedure, such as but not limited to brachioplasty and abdominoplasty, that removes fat and skin together. For example, a dermal-lipectomy specimen is an adipose-containing tissue.

A “dermatologically acceptable vehicle” as used herein refers to entities and compositions that are physiologically tolerable and do not typically produce an allergic or similar untoward reaction when administered to a subject that is suitable for topical application to the keratinous tissue and is compatible with the fat lysate described herein. As used herein, “dermatologically acceptable” means that the ingredients which the term describes are suitable for use in contact with the skin without undue toxicity, incompatibility, instability, irritation, allergic response, and the like. The vehicle can be in a wide variety of forms, including, but not limited to, oil-in-water emulsions, water-in-oil emulsions, water-in-silicone emulsions, silicone-in-water emulsions, water-in-oil-in-water, and oil-in-water-in-oil emulsions, and oil-in-water-in-silicone emulsions. Suitable surfactants include anionic, cationic, amphoteric, zwitterionic and non-ionic, including those listed in U.S. Pat. No. 6,197,319. The International Cosmetic Ingredient Dictionary and Handbook (10th Edition, 2004), published by the Cosmetic, Toiletries & Fragrance Association, describes a wide variety of non-limiting cosmetic and dermatopharmaceutical ingredients commonly used in the skin care industry, which are suitable for use in combination with the disclosed fat lysate, the disclosure of which is incorporated by reference herein in its entireties for its teachings of dermatopharmaceutical ingredients. Examples of these ingredients include: antioxidants, anti-inflammatory agents, anti-acne agents, antimicrobial agents, astringents, humectants, moisturizers, pH adjusters, skin bleaching/lightening agents, skin soothing/healing agents and agents that help decrease the appearance of signs of aging.

“Keratinous tissue” includes keratin-containing layers disposed as the outermost protective covering of mammals and includes, but is not limited to, skin, hair and nails. As used herein, a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

As used herein, “rejuvenate” is meant a full or partial reversal of at least one phenotype typical of an aging cell Skin rejuvenation can include one or more of the following: reducing the appearance of fine lines and wrinkles; reducing deep wrinkles; enhancing skin tone and elasticity; reducing skin blemishes and/or age spots; reducing skin roughness; and producing a younger looking skin.

### Compositions

Cosmetic compositions comprising lysed adipose tissue are described herein. Lysed adipose tissue can comprise lysed adipocytes, preadipocytes, fibroblasts, endothelial cells, multipotent stem cells, and combinations thereof. In some examples, the lysed adipose tissue can comprise intracellular, extracellular, and interstitial contents of adipocytes. The intracellular components can include adipokines and/or adipose-derived hormones. Adipokines and adipose-derived hormones can include, but are not limited to leptin, adiponectin, estrogen, resistin, visfatin, adipsin, and apelin. In some aspects, the leptin, adiponectin, or a combination thereof can be the active ingredients in the disclosed compositions. Other components of the adipose tissue can contribute to the positive effect of the lysed adipose tissue compositions. In some examples, the lysed adipose tissue can comprise vasculature and connective tissues. The vasculature and connective tissues can include stroma. In some aspects, the lysed adipose tissue can comprise stromal vascular fraction (SVF) of cells including preadipocytes, fibroblasts, vascular endothelial cells and/or a variety of immune cells (i.e. adipose tissue macrophages (ATMs)).

The adipose tissue can be derived from any suitable source. The adipose tissue can be brown or white adipose tissue, derived from subcutaneous, omental/visceral, mammary, gonadal, or other adipose tissue site. The adipose tissue can be from any organism having adipose or fat tissue. In some aspects, the adipose tissue can be mammalian, such as human adipose tissue. In some aspects, the adipose tissue is limited to subcutaneous fat and excludes fat taken from the breast or other non-subcutaneous sources (such as intraabdominal). In some aspects, the adipose tissue can be fish adipose tissue. The adipose tissue can be derived in vitro, for example, from a cell culture. In some examples, adipocytes, preadipocytes, fibroblasts, endothelial cells, multipotent stem cells, adipokines, or adipose derived hormones can be obtained from preadipocyte cell lines, adipocyte cell lines, or multipotent stem cell lines. The adipose tissue can be obtained from a plant source. For example, adipokines and/or adipose derived hormones can be obtained from plants such as vegetables, pulse (legume), avocado, nuts, and such the like. In some examples, the adipokines and/or adipose derived hormones can be a plant-derived homolog. For example, osmotin is a plant derived homolog of mammalian adiponectic, which can be used in the cosmetic compositions. The adipose tissue can be synthetic. In some examples, adipokines or adipose derived hormones can be synthetic.

The cosmetic composition can comprise the lysed adipose tissue in an amount of from about 0.001% to about 80% by weight of the composition. In some aspects, the compositions can comprise from about 0.001% to about 50%, from about 0.001% to about 25%, from about 1% to about 10%, from about 2% to about 5% by weight of lysed adipose tissue. The concentration of the lysed adipose tissue in the cosmetic compositions can be about 50 μg/mL or greater. In some aspects, the concentration of the lysed adipose tissue in the cosmetic compositions can be from about 50 μg/mL to about 1 mg/mL, for example from about 200 μg/mL to about 1 mg/ml or from about 50 μg/mL to about 500 μg/mL.

### Cosmetic Compositions

Cosmetic compositions comprising the lysed adipose tissue and a dermatological acceptable vehicle are also described herein. The dermatological acceptable vehicle can include an emollient, a diluent, a solubilizing or emulsifying agent, a solvent, a propellant, a powder, or combinations thereof.

Emollients, as used herein, refer to materials used for the prevention or relief of dryness, as well as for the protection of the skin. Examples of emollients that can be used as suitable dermatological carriers include, but are not limited to stearyl alcohol, glycerol monoricinoleate, glycerol monostearate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanylalcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, tallow, lard, olive oil, palm kernal oil, rapeseed oil, safflower seed oil, evening primrose oil, soybean oil, sunflower seed oil, avocado oil, olive oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, and myristyl myristate. More than one emollient may be included in the composition.

Examples of propellants that can be used as suitable dermatological carriers include, but are not limited to, a liquefiable gas or a halogenated propellant. Specific examples of propellants include dimethyl ether, trichlorofluoromethane, diclorodifluoromethane, dichlrorotetrafluroethane, monochlorodifluoromethane, trichlorotrifluoroethane, propane, butane, carbon dioxide, nitrous oxide, or combinations thereof. The amount of propellant in the composition can be from about 10% to about 60% by weight of the composition.

Examples of solvents that can be used as suitable dermatological carriers include, but are not limited to ethyl alcohol, methylene chloride, isopropanol, acetone, ethylene glycol monoethyl ether, diethylene glycol monobutyl ether, diethylene glycol monoethyl ether, dimethyl sulphoxide, dimethyl formamide, tetrahydrofuran, propylene glycol, butylene glycol, dipropylene glycol, ethoxylated or propoxylated diglycols, cyclic polyols, or combinations thereof.

Examples of emulsifiers that can be used as suitable dermatological carriers include nonionic, anionic or cationic emulsifiers. Suitable emulsifiers are disclosed, for example, in McCutcheon's Detergents and Emulsifiers, North American Edition, pp. 317-324 (1986), and the ICI Handbook, pp. 1673-1686. Single emulsion skin care preparations, such as lotions and creams, of the oil-in-water type and water-in-oil type are well known in the cosmetic art and are useful in the present disclosure. Multiphase emulsion compositions, for example the water-in-oil-in-water type, as disclosed in U.S. Pat. Nos. 4,254,105 and 4,960,764, which are incorporated by reference herein in their entireties for their teachings of emulsions, can also be useful. In general, such single or multiphase emulsions contain water, emollients, and emulsifiers as essential ingredients. The compositions can comprise from about 1% to about 10% (e.g., from about 2% to about 5%) of an emulsifier(s). The emulsion can be a microemulsion or a nanoemulsion.

Examples of powders that can be used as suitable dermatological carriers include, but are not limited to chalk, talc, fullers earth, kaolin, starch, gums, colloidal silica sodium polacrylate, tetre alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate, or combinations thereof.

In some examples, the cosmetic composition can comprise the lysed adipose tissue and a dermatological acceptable vehicle, wherein the dermatological acceptable vehicle can include water, mineral oil, petrolatum, ceresin, lanolin alcohol, methylchloroisothiazolinone, methylisothiazolinone, and combinations thereof. In some examples, the cosmetic composition can comprise an adipokine and/or a adipose derived hormone and a dermatological acceptable vehicle, wherein the dermatological acceptable vehicle can include water, mineral oil, petrolatum, ceresin, lanolin alcohol, methylchloroisothiazolinone, methylisothiazolinone, and combinations thereof.

The dermatological acceptable vehicle can be present in an amount of from about 10 wt % to about 99.999 wt %, for example about 20 wt % to about 99.999 wt %, about 40 wt % to about 90 wt % of the cosmetic composition. In some aspects, the cosmetic composition can also comprise water up to about 98% volume, for example, about 5 to about 80% volume of the cosmetic composition.

### Other Agents in the Formulations

The cosmetic compositions described herein can contain additional oil-soluble materials and/or water-soluble materials conventionally used in compositions for use on skin, hair, and nails. Specific examples of other suitable agents that can be included in the cosmetic composition, include a moisturizing agent, humectants, surface active agents, binding agents, thickeners, viscosity modifiers, buffers, preservatives, neutral or cationic lipids, lipid complexes, liposomes, polymers, sunscreen agents, lubricants, antioxidants, proteins, amino acids, fragrances, perfumes, oils, natural extracts such as plant extracts, butters, vitamins, pH adjusting agents, absorbents, other dermatological acceptable excipients, and combinations thereof.

The cosmetic compositions can comprise a moisturizing agent or a humectant. In some examples, the cosmetic composition can be combined with other ingredients such as moisturizers, cosmetic adjuvants, anti-oxidants, depigmenting agents, darkening agents, anti-aging agents, hair removal agents, hair styling agents, nail styling agents, sunscreens, surfactants, bleaching agents, foaming agents, conditioners, humectants, fragrances, colorants, viscosifiers, buffering agents, preservatives, and the like and mixtures thereof. Skin-care compositions including these components should be formulated so as not to affect the activity of the lysed adipose tissue.

Examples of humectants include glycerol, sorbitol, propylene glycol, ethylene glycol, 1,3-butylene glycol, polypropylene glycol, xylitol, malitol, lactitol, allantoin, acetamine MEA, oat protein, hyaluronic acid, and the like. They can be used either singly or in combination. Preservatives can also be included in the cosmetic compositions.

Preservatives are useful for substantially preventing microbial decomposition. Examples of suitable preservatives include phenoxyethanol and parabens such as methyl-paraben, ethyl-paraben, and propyl-paraben; salicylic acid, chlorhexidine hydrochloride, phenoxyethanol, sodium benzoate, methyl para- hydroxybenzoate, ethyl para-hydroxybenzoate, propyl para-hydroxybenzoate, butyl para- hydroxybenzoate, isothiazolones and the like. Other examples of preservatives are listed on pages 1654-55 of the International Cosmetic Ingredient Dictionary and Handbook, eds. Wenninger and McEwen (CTFA, 7th ed., 1997), hereinafter referred to as the “Cosmetic Handbook.” The composition can comprise from about 0.01% to about 20%, by weight (more preferably, from about 0.5% to about 5%, by weight) of preservative. Microbial contamination can also be eliminated by gamma irradiation or microfiltration, or by brief heat treatments that do not result in the elimination of protease inhibitory activity.

Examples of fragrances and odor masks that can be included in the disclosed compositions include menthol, anethole, carvone, eugenol, limonene, ocimene, n-decylalcohol, citronellol, a-terpineol, methyl salicylate, methyl acetate, citronellyl acetate, cineole, linalool, ethyl linalool, vanillin, thymol, spearmint oil, peppermint oil, lemon oil, orange oil, sage oil, rosemary oil, cinnamon oil, pimento oil, cinnamon leaf oil, perilla oil, wintergreen oil, clove oil, eucalyptus oil and the like.

Examples of surface active agents that can be included in the disclosed compositions include sodium alkyl sulfates, e.g., sodium lauryl sulfate and sodium myristyl sulfate, sodium N-acyl sarcosinates, e.g., sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate and N-acyl glutamates, e.g., N-palmitoyl glutamate, N-methylacyltaurin sodium salt, N-methylacylalanine sodium salt, sodium α-olefin sulfonate and sodium dioctylsulfosuccinate; N-alkylaminoglycerols, e.g., N-lauryldiaminoethylglycerol and N-myristyldiaminoethylglycerol, N-alkyl-N-carboxymethylammonium betaine and sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, PIURONIC™ type surface active agent, and polyoxyethylenesorbitan aliphatic acid esters such as polyoxyethylenesorbitan monooleate, polyoxyethylenesorbitan monolaurate, and combinations thereof.

Examples of the binder or thickener include cellulose derivatives such as alkali metal salts of carboxymethylcellulose, methyl cellulose, hydroxyethyl cellulose and sodium carboxymethylhydroxyethyl cellulose, alkali metal alginates such as sodium alginate, propylene glycol alginate, gums such as carrageenan, xanthan gum, tragacanth gum, caraya gum and gum arabic, and synthetic binders such as polyvinyl alcohol, polysodium acrylate and polyvinyl pyrrolidone. Thickening agents that can be added to the compositions to alter viscosity include other polymers such as polyacrylates (e.g., polyacrylamide). Other examples of viscosity modifying agents are listed on pages 1692-97 of the Cosmetic Handbook, which is incorporated by reference herein in its entirety for its teachings of viscosity modifying agents. To achieve the appropriate viscosity, the cosmetic compositions may comprise from about 0.01% to about 20%, by weight (e.g., from about 0.1% to about 5%, by weight) of a thickening agent.

Coloring agents and fragrances can also be included in the compositions comprising lysed adipose tissue disclosed herein.

In some aspects, the cosmetic compositions further comprise a second cosmetically active agent in addition to the lysed adipose tissue. A “cosmetically active agent” is a compound (e.g., a synthetic compound or a compound isolated from a natural source or a natural extract) that has a cosmetic or therapeutic effect on the skin, hair, or nails, including, but not limiting to, lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, firming agents, anti-callous agents, and agents for hair, nail, and/or skin conditioning.

The cosmetic compositions disclosed herein can be applied prior to, concurrently with or after other active ingredients or compositions to enhance their effect.

Antioxidants and/or chelating agents can be used to increase shelf life and stability of the disclosed cosmetic compositions. Antioxidants can be added both for formulation stabilization and for biological efficacy. Antioxidant compounds and their derivatives include, but are not limited to, water-soluble antioxidants such as sulfhydryl compounds and their derivatives (e.g., sodium metabisulfite and N-acetyl-cysteine), lipoic acid and dihydrolipoic acid, resveratrol, acetyl-cysteine (INIFERINE™) or lactoferrin, and ascorbic acid and ascorbic acid derivatives (e.g., ascorbyl palmitate and ascorbyl polypeptide). Oil-soluble antioxidants suitable for use in the compositions include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the disclosed cosmetic compositions, include, but not limited to, extracts containing flavonoids and isoflavonoids and their derivatives (e.g., genistein and diadzein), extracts containing resveratrol and the like. Examples of such natural extracts include grape seed, green tea, pine bark, propolis, and legume extracts. Other examples of antioxidants can be found on pages 1612-1613 of the Cosmetic Handbook. The cosmetic compositions disclosed herein can comprise the antioxidant in an amount of from about 0.001% to about 20%, by weight (e.g., from about 0.01% to about 10% by weight) of the composition.

In some examples, at least one oil-soluble antioxidant can be present in the cosmetic compositions. The antioxidants can be utilized in a stabilizing effective amount and may range in total from about 0.001 to 10% based on the weight of the total composition, for example from about 0.005 to about 5%. The oil-soluble antioxidants which are useful in the compositions include butylated hydroxytoluene (BHT), ascorbyl palmitate, butylated hydroxanisole (BHA), phenyl-α-naphthylamine, hydroquinone, propyl gallate, nordihydroguiaretic acid, and mixtures thereof as well as any other known oil-soluble antioxidant compatible with the other components of the disclosed cosmetic compositions.

A water-soluble antioxidant can be present in a water phase of the disclosed cosmetic compositions. The water-soluble antioxidants can include ascorbic acid, sodium metabisulfite, sodium bisulfate, sodium thiosulfite, sodium formaldehyde sulfoxylate, isoascorbic acid, thioglyerol, thiosorbitol, thiourea, thioglycolic acid, cysteine hydrochloride, 1,4-diazobicyclo-(2,2,2)-octane and mixtures thereof as well as any other known water-soluble antioxidant compatible with the other components of the disclosed cosmetic compositions.

Chelating agents are also useful in assisting the stabilization of the disclosed cosmetic compositions. Examples of chelating agents include EDTA and derivatives thereof (e.g., disodium EDTA and dipotassium EDTA), INIFERINE™, lactoferrin, and citric acid. Other examples of chelating agents are listed on page 1626 of the Cosmetic Handbook, which is incorporated by reference herein in its entirety for its teachings of chelating agents. The disclosed cosmetic compositions can comprise the chelating agent in an amount of from about 0.001% to about 20%, by weight (e.g., from about 0.01% to about 10% by weight) of the composition.

Other active ingredients such as sunscreen materials can be utilized in the disclosed cosmetic compositions provided that they are physically and chemically compatible with the other components of the compositions. Sunscreens can include organic or inorganic sunscreens, such as methoxyoctylcinnamate and other cinnamate compounds, titanium dioxide and zinc oxide and the like.

Various irritancy mitigants can be added to the disclosed cosmetic compositions. Irritancy mitigants such as a-bisabolol, panthenol, allantoin, ginkgo biloba, stearoyl glycerrhetinic acid (licorice extract), tea tree oil, butchers' broom, calendula, ginseng and the like can be added.

Other ingredients can include agents that assist in protecting the skin from aging, such as sunscreens, anti-oxidant vitamins such as ascorbic acid, vitamin B, biotin, pantothenic acid, vitamin D, vitamin E and vitamin C, and sodium bisulfite. Yeast extract, gingko biloba, bisabolol, panthenol, alpha hydroxy acids and oligosaccharides such as melibiose are among other ingredients which assist in preventing aging of the skin by such means as irritation mitigation, oxidation mitigation, healing, affecting retinoid metabolism and inhibiting the production of elastase.

The disclosed cosmetic compositions can also contain depigmenting agents. What is meant by depigmentation is the lightening of the color of an area of skin, including but not limited to, the global lightening of the user's skin tone/complexion (e.g., the face, hands, or whole body, which is uneven as a result of aging skin, or darker than desired because of ethnicity or pathology, and the like), the evening of skin color tone, or the specific lightening of age spots, freckles, or darker pigmented areas such as, but not limited to, post-inflammatory hyper-pigmentary lesions.

Examples of depigmenting agents include, but are not limited to, lipoic acid, dihydrolipoic acid, resveratrol, ascorbic acid, kojic acid, hydroquinone, isoflavones, retinoids (e.g., retinol, retinoic acid, and retinol palmitate), tyrosinase inhibitors, melanosome transfer inhibitors, and selective cytotoxic agents for melanocytes, or natural extracts, e.g., licorice extract, gatuline A (pilewort extract), and micromerol (butylene glycol and apple extract), providing these activities. The amount of the depigmenting agent used will depend on the activity of the compound, and can be in an amount from about 0.001% to about 20%, by weight (e.g., from about 0.01% to about 10%, by weight) of the composition.

Other skin color evening ingredients, such as skin darkening or sunless tanning agents, can also be effective in the cosmetic compositions disclosed herein.

The disclosed cosmetic compositions can also contain compounds that enhance the feel of the composition on the skin of the user. Examples of such compounds include, but are not limited to, oils, silicones (e.g., siloxane polymers such as dimethicone) and skin-conditioning agents such as emollients, and humectants. Examples of such skin conditioning agents may be found of pages 1656-1670 of the Cosmetic Handbook, which is incorporated by reference herein in its entirety for its teachings of skin conditioning agents.

Compositions which assist in the reduction of lines and wrinkles can also be added to the cosmetic compositions. For example, alpha hydroxy acids, hyaluronic acid, Gatuline R (fagus silvitica extract), pigments and scattering aids such as zinc oxide and titanium dioxide can be used in the disclosed cosmetic compositions.

Anti-inflammatory agents can also be used in the compositions disclosed herein. Not only can these agents assist in mitigating irritation, they can assist in treating wrinkles and lines in the skin. Examples of steroidal anti-inflammatory agents, include but are not limited to, corticosteroids such as hydrocortisone, hydroxyltriamcinolone, alpha-methyl dexamethasone, dexamethasone-phosphate, beclomethasone dipropionate, clobetasol valerate, desonide, desoxycorticosterone acetate, dexamethoasone, dichlorisone, deflorasonediacetate, diflucortolone valerate, fluadronolone, fluclarolone acetonide, fludrocortisone, flumethasone pivalate, fluosinolone acetonide, fluocionide, flucortine butylester, fluocortolone, flupredidene (flupredylidene) acetate, flurandronolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenalone acetonide, medrysone, amciafel, amcinafide, betamethasone and its esters, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone and mixtures thereof may be used. Preferably, hydrocortisone or natural extracts with similar activity can be used.

Nonsteroidal anti-inflammatory agents can also be employed in the disclosed cosmetic compositions, such as salicylates, acetic acid derivatives, fenamates, propionic acid derivatives and pyrazoles or mixtures thereof. Other synthetic and natural anti-inflammatory agents can also be used.

Additional active ingredients having topical activity can be utilized in the disclosed cosmetic compositions. Azole-type anti-fungal and anti-bacterial agents can be employed in the disclosed cosmetic compositions in their base form. For example, ketoconazole, miconazole, itraconazole, elubiol, and like related imidazole antifungals and antibacterials are useful in the topical formulations.

It can be readily appreciated that a transdermal route of administration of the cosmetic compositions can be enhanced by use of a dermal penetration enhancer, e.g., such as enhancers described in U.S. Pat. No. 5,164,189, U.S. Pat. No. 5,008,110, and U.S. Pat. No. 4,879,119. In some aspects, the disclosed compositions can be delivered in a controlled release system, such as using a transdermal patch, liposomes, or other modes of administration. In some aspects, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)].

### Liposomal Formulations

Liposomal agents can be used to increase shelf life and stability of the lysed adipose tissue in the cosmetic compositions. Thus, the cosmetic composition can be formulated as a liposomal formulation. In some examples, the lysed adipose tissue can be encapsulated or contained within liposomes. The liposome preparation can then be incorporated into the dermatological vehicle (e.g., a gel or an oil-in-water emulsion) in order to produce the topical liposomal formulation. Examples of suitable liposomes include unilamellar, multilamellar, and paucilamellar liposomes.

The liposome in the cosmetic composition can be ionic or nonionic. In some aspects, the liposomes can include negatively charged phospholipids. Negatively charged phospholipids can help prevent aggregation of the liposomes. The phospholipids can be from natural origin including, but not limited to eggs and soybean. In some examples, the liposomes can contain about 5 to about 15 mole % of negatively charged phospholipids, such as phospatidylglycerol, phospatidylserine, phospatidylinositol, lecithins, phosphatidyl ethanol amines, or sphingomyelins.

The liposome in the composition can be nonionic. In some examples, the liposome can contain (a) glycerol dilaurate; (b) compounds having the steroid backbone found in cholesterol; and (c) fatty acid ethers having from about 12 to about 18 carbon atoms. In some aspects, the liposome can comprise glycerol dilaurate, cholesterol, polyoxyethylene-10-stearyl ether, and polyoxyethylene-9-lauryl ether. In some aspects, these ingredients are in a ratio of about 38:12:33:17.

In some aspects, the liposomes can include epidermal lipids. Other liposomal compositions for topically application are described in Mezei, M., “Liposomes as a Skin Drug Delivery System”, Topics in Pharmaceutical Sciences (D. Breimer and P. Speiser, eds.), Elsevier Science Publishers B. V., New York, N.Y., 1985, pp. 345-358, PCT Patent Application No. WO96/31194, Niemiec, et al., 12 Pharm. Res. 1184-88 (1995), and U.S. Pat. No. 5,260,065, which are incorporated by reference herein in their entireties for their teachings of liposomal compositions and methods of skin drug delivery.

The liposome can include agents that protect the lysed adiposed tissue from free-radical and lipid-peroxidative damage (e.g., during storage). Non-limiting examples of such lipid-protective agents include fat-soluble antioxidants, such as tocopheryl acetate, retinyl palmitate, butylated hydroxytoluene, butylated hydroxyanisole, ascorbyl palmitate and mixtures thereof.

The liposomes can be present in the cosmetic composition in an amount, based upon the total volume of the composition, of from about 5 mg/ml to about 100 mg/mL such as from about 10 mg/mL to about 50 mg/mL.

### Topical Formulations

The cosmetic composition can be formulated for topical application to skin. The topical formulation can comprise the lysed adipose tissue in a topically applicable base. The topically applicable base can be in the form of a lotion, cream, gel, hydrogels, serum, colloid, spray, pastes, foams, powders, solution, emulsion, or films. The topically formulation can be included or made into a variety of product types including, but are not limited to, lotion including skin lotion, milk lotion, moisture lotion, cleansing lotion, nutrient lotion, massage cream, nutrient cream, moisture cream, hand cream, foundation, cleansing foam, cleansing cream, body cleanser, skin softener, skin toner, astringent, ointment, cleansing liquid wash, shaving creams, moisturizer, mask, mask wipe, patches, adhesive bandages, or any other beauty product. Make-up, such as lipsticks are also suitable form of the compositions.

In some aspects, the topical formulation can be a skin lotion or a cream. The lotion can comprise from about 1% to about 20% (e.g., from about 5% to about 10%) of an emollient(s) and from about 50% to about 90% (e.g., from about 60% to about 80%) of water. The cream can comprise from about 5% to about 50% (e.g., from about 10% to about 20%) of an emollient(s) and from about 45% to about 85% (e.g., from about 50% to about 75%) of water. The lotion and/or cream can be formulated as an emulsion. Such lotion can comprise from about 0.5% to about 5% of an emulsifier(s). Such cream can comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

In some aspects, the topical formulation can be an ointment. The ointment can comprise a simple base of animal or vegetable oils or semi-solid hydrocarbons. An ointment can comprise from about 2% to about 10% of an emollient(s) and from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697, which is incorporated by reference herein in its entirety for its teachings of thickening agents.

In some aspects, the topical formulation can be a gel (e.g., an aqueous, alcohol, alcohol/water, or oil gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents.

The topical formulations can also be formulated as a solid formulation (e.g., a wax-based stick, soap bar composition, powder, or a wipe containing powder).

### Methods of Preparing Lysed Adipose Tissue and Topical Formulations

Methods of preparing lysed adipose tissue (fat lysate) and topical formulations comprising the lysed adipose tissue are described herein. The method can include obtaining or removing adipose tissue from any suitable source. In some examples, the adipose tissue can be obtained from or removed by a lipectomy procedure which excises or extracts subcutaneous fat tissue, including, but not limited to, suction assisted lipectomy (liposuction), dermal lipectomy procedures used for body contouring (such as abdominoplasty, brachioplasty, thigh-plasty, belt-lipectomy, buttock lifting), and isolated lipectomy (direct excision or suction assisted) from a subject. In some aspects, the fat is limited to subcutaneous fat and excludes fat taken from the breast or other non-subcutaneous sources (such as intraabdominal). In some aspects, the fat tissue is marked for disposal and not pathological analysis of any type. In some aspects, the adipokine, adipose derived hormone, or a homolog thereof can be obtained from a plant source. The method of obtaining the fat tissue from a plant source can include lysing the plant source followed by extraction using any suitable method known to those of skill in the art for extracting proteins or fats from cells. Adipocytes, preadipocytes, fibroblasts, endothelial cells, multipotent stem cells, adipokines, or adipose derived hormones can be obtained in vitro, for example from a cell culture. Moremo-Navarette et al. (Adipose Tissue Biology, M. E. Symonds (ed.), Springer Science Business Media, LLC, 2012; Chpt 2:17-38), which is incorporated by reference herein in its entirety for its teachings of methods of obtaining cell tissue, describes methods of obtaining adipocytes, preadipocytes, fibroblasts, endothelial cells, and multipotent stem cells from preadipocyte cell lines, adipocyte cell lines, and multipotent stem cell lines.

The adipose tissue can be lysed using known chemical or mechanical techniques of lysing. For example, lysis can occur using common lysis buffers such as, but not limited to, RIPA buffer and hypertonic saline. Mechanical mechanisms include but are not limited to mechanical disruption, liquid homogenization, sonication and freeze-thawing. The lysed adipose tissue can comprise the content and the cellular debris from adipocytes and all other cells found in the tissue, including vasculature and connective tissue. In some examples, the lysed adipose tissue does not comprise any live cells. Lysing the adipose tissue can eliminate any live cells. The elimination of live cells can be confirmed using standard cell viability assays, such as trypan blue staining.

The lysed adipose tissue can be filtered or purified before combining with any other ingredient in the cosmetic composition, for example, the topically applicable base. For example, the lysed adipose-containing tissue can be purified by filtering to remove debris. In some aspects, 0.2 micrometer filter paper can be used. In some examples, the lysed adipose tissue is not filtered or purified before use.

Methods of preparing the fat lysate topical formulation can include combining the lysed adipose-containing tissue with a topically applicable base. The step of combining the lysed adipose tissue with a topically applicable base can include a wide range of concentrations. For example, disclosed are methods of preparing a fat lysate topical formulation comprising a) removing adipose-containing tissue from a subject or obtaining adipose-containing tissue removed from a subject; b) lysing the adipose-containing tissue; and c) combining the lysed adipose tissue with a topically applicable base, wherein every 1 mL of topically applicable base is combined with about 200μg of lysed adipose tissue. The formulation can also include about 50, about 100, about 150, about 200, about 250, about 300, about 350, about 400, about 450, about 500, about 550, about 600, about 650, about 700, about 750, about 800, about 850, about 900 or about 950 μg of lysed adipose tissue for every 1 mL of topically applicable base. In some aspects about 1, about 2, about 3, about 4, or about 5 mg of lysed adipose tissue can be combined with 1 mL of topically applicable base. The more problematic the skin area is that is being treated, the more concentrated the lysed adipose tissue formulation can be. For example, a subject can begin treatment with a highly concentrated formulation (i.e. about 1 to about 5 mg/mL) and as the skin rejuvenates

In some examples, the lysed adipose tissue can be contained in a liposomal agent before combining with a topically applicable base. Methods of preparing a liposomal formulation can include combining the lysed adipose tissue with a phospholipid, such as dipalmitoylphosphatidyl choline, cholesterol and water. An example of a method for producing liposomes is described in Mezei & Gulasekharam, “Liposomes—A Selective Drug Delivery System for the Topical Route of Administration; Gel Dosage Form”, Journal of Pharmaceutics and Pharmacology, Vol. 34 (1982), pp. 473-474. Those of skill in the art may make suitable modifications of the method described therein.

### Methods of Rejuvenating Skin

Methods of rejuvenating skin comprising administering to a subject a cosmetic composition comprising lysed adipose tissue. The cosmetic composition administered to the subject can be any of the cosmetic compositions described herein. For example, the method of rejuvenating skin can involve administering a cosmetic composition containing lysed adipose tissue, wherein the lysed adipose tissue comprises the intracellular, extracellular, and interstitial contents from adipocytes. The intracellular content can contain adipokines including, but not limited to, leptin and adiponectin. The adipose tissue can be derived from the subject. Thus, the lysed adipose tissue can be autologous to the subject being administered the cosmetic composition comprising the lysed adipose tissue. In some aspects, the adipose tissue can be derived from a subject other than the subject being administered the cosmetic composition. Thus, the adipose tissue can be autologous, allogeneic or xenogeneic to the subject to whom the cosmetic composition comprising the lysed adipose tissue is being administered to.

In some examples, administering the cosmetic composition comprising the lysed adipose tissue can include topical administration. In some aspects, the cosmetic composition can be administered as a lotion, cream, gel, stick, spray, colloid, ointment, cleansing liquid wash, solid bar, shampoo, paste, foam, powder, mousse, shaving cream, wipe, patch, wound dressing, adhesive bandage, hydrogel, or film.

Methods of rejuvenating skin can comprise administering to a subject the cosmetic composition comprising lysed adipose tissue, wherein the composition can be administered daily. In some aspects, the cosmetic composition can be administered in any routine or regularly recurring frequency. In some aspects, the cosmetic compositions can be administered irregularly. For example, the cosmetic compositions can be administered hourly, daily, weekly, or monthly on a routine basis. On the other hand, the cosmetic compositions can be administered every few days or every few months depending on the need. In some aspects, the subject being administered the composition can assess their need for administration.

The concentration of the lysed adipose tissue in the cosmetic compositions for rejuvenating the skin can be 50 μg/mL or greater. In some aspects, the concentration of the lysed adipose tissue in the cosmetic compositions can be from about 50 μg/mL to about 500 μg/mL. In some aspects a higher concentration can be used. For example, an area of skin such as perioral and peri-orbital rhytids can use concentrations of the cosmetic composition ranging from 200 μg/mL to 1 mg/mL.

Rejuvenating the skin can include improving or restoring aged or damaged skin, improving the appearance or texture of the skin, or enhancing the skin. Rejuvenating the skin can result in fewer wrinkles, fuller or plumper skin, less shriveled skin, less flabby skin, less thin skin, improved elasticity and/or tonus of the skin, less dull skin without a glow, decreased pigmentation spots on the skin, more youthful appearing skin. Disclosed are methods of rejuvenating skin comprising administering to a subject a cosmetic composition comprising lysed adipose tissue, wherein the rejuvenating can results in fewer wrinkles, fuller skin, plumper skin, or a combination thereof.

Measuring the effects of the lysed adipose tissue composition on the skin can be performed with a variety of known techniques. Besides determining the effects based on visual appearance, topical imaging devices can be used. For example, optical coherence tomography can be used to image the skin.

### Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for preparing fat lysate topical formulations, the kit comprising a topically applicable base. The kits also can contain lysis buffer for lysing the adipose-containing tissue. In some aspects, the kits have some type of cooling mechanism such as but not limited to a cold pack or dry ice.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present disclosure, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

Protein Isolation from Human Adipose Tissue and Formulation of Fat-Lysate Cream

The volunteer test subject was a 37-year-old, 80 kg otherwise healthy male subject. Suction-assisted lipectomy was performed on the lower abdomen after local anesthesia was achieved with subcutaneous injection of 3 mL of lidocaine with 1:100,000 epinephrine. A total of 3 cubic centimeters of lipoaspirate was harvested and immediately placed on ice. All subsequent steps were performed at 4° C. Using a sterile razor blade, adipose tissue was cut into 250 mg sections. Each section was then placed immediately into a 1.7 mL microtube containing 200 uL of RipA Buffer (Thermo Scientific, 25 mM Tris·HCl pH 7.6, 150 mM NaCl, 1% NP-40, 1% sodium deoxycholate, 0.1% SDS) and kept on ice for 30 minutes. Each sample was then sonicated using a sonic dismembrator (Fischer Scientific, Model 100) at a power of 5 for 30 seconds and placed on ice for 30 minutes before being centrifuged at 15,000 g for 20 minutes.

The supernatant was collected and protein concentration was determined using a Pierce BCA Protein Assay Kit (#23227). Samples were diluted to a protein concentration of 2000 μg/mL in RipA buffer and 200 μL (containing 400 ug of total protein) was added drop wise to 2 cubic centimeters of cream base (Eucerin Original Healing Cream, chosen for its basic ingredients: water, petrolatum, mineral oil, ceresin, lanolin alcohol, methylchloroisothiazolinone, methylisothiazolinone) with constant mixing. This fat-lysate cream was stored at ambient temperature.

### Testing of Fat-Lysate Cream

The fat-lysate cream was compared to a control mixture of cream prepared exactly as described above, except for the addition of fat lysate. The control cream was mixed with the same buffers and stored as described above for the fat-lysate cream. To test the creams, they were applied twice daily to the dorsum of the hand. The control cream was applied to the left hand and the fat-lysate cream was applied to the right hand. The test subject was the donor of the fat used to produce the fat-lysate cream described above. On the day prior to cream application, three plastic surgical professionals evaluated the donor's hands. On day 0, the application of the creams to the dorsum of each hand was started and continued for 10 days. At the completion of this period, the same plastic surgical professionals were asked to re-evaluate the treated areas, blinded to which side was treated with fat-lysate cream or control. In all three evaluations, the right hand was rated to be superior in appearance, fullness, smoothness, and noted to have fewer wrinkles.

What is claimed is:

1. A cosmetic composition, comprising: lysed adipose tissue and a cosmetically acceptable carrier.

2. The composition of claim 1, wherein the lysed adipose tissue comprises the intracellular, extracellular, and interstitial contents from adipocytes.

3. The composition of any one of the preceding claims, wherein the intracellular content comprises adipokines.

4. The composition of any one of the preceding claims, wherein the adipokines are leptin and adiponectin.

5. The composition of any one of the preceding claims, wherein the lysed adipose tissue comprises lysed adipose tissue stroma.

6. The composition of any one of the preceding claims, wherein the lysed adipose tissue comprises adipose derived stem cells.

7. The composition of any one of the preceding claims, wherein the cosmetically acceptable carrier is a dermatologically acceptable vehicle.

8. The composition of any one of the preceding claims, wherein the dermatological vehicle is selected from the group consisting of mineral oil, petrolatum, ceresin, lanolin alcohol, methylchloroisothiazolinone, methylisothiazolinone, and combinations thereof.

9. The composition of any one of the preceding claims, wherein the cosmetic composition further comprises a moisturizing agent or a humectant.

10. The composition of any one of the preceding claims, wherein the cosmetic composition is a lotion, cream, gel, serum, or emulsion.

11. The composition of any one of the preceding claims, wherein the cosmetic composition is a cleanser, a toner, a moisturizer, or a mask.

12. The composition of any one of the preceding claims, wherein the cosmetic composition comprises 0.001% to 80% by weight of lysed adipose tissue.

13. The composition of any one of the preceding claims, wherein each 1 mL of the cosmetic composition comprises 200 μg of lysed adipose tissue.

14. The composition of any one of the preceding claims, wherein the lysed adipose tissue does not comprise any live cells.

15. A cosmetic composition, comprising: an adipose derived hormone and a cosmetically acceptable carrier.

16. The composition of any one of the preceding claims, wherein the adipose derived hormone includes leptin, adiponectin, and combinations thereof.

17. A method of rejuvenating skin, comprising: topically administering to a subject a cosmetic composition comprising lysed adipose tissue and a cosmetically acceptable carrier.

18. The method of any one of the preceding claims, wherein the lysed adipose tissue comprises the intracellular, extracellular, and interstitial contents from adipocytes.

19. The method of any one of the preceding claims, wherein the intracellular content comprises adipokines.

20. The method of any one of the preceding claims, wherein the adipokines are leptin and adiponectin.

21. The method of any one of the preceding claims, wherein the lysed adipose tissue comprises lysed adipose tissue stroma.

22. The method of any one of the preceding claims, wherein the lysed adipose tissue comprises adipose derived stem cells.

23. The method of any one of the preceding claims, wherein the adipose tissue is obtained from the subject.

24. The method of any one of the preceding claims, wherein the lysed adipose tissue is autologous, allogeneic, or xenogeneic to the subject.

25. The method of any one of the preceding claims, wherein the lysed adipose tissue is autologous to the subject.

26. The method of any one of the preceding claims, wherein the cosmetically acceptable carrier is a dermatologically acceptable vehicle.

27. The method of any one of the preceding claims, wherein the dermatologically acceptable vehicle comprises water, mineral oil, petrolatum, ceresin, lanolin alcohol, methylchloroisothiazolinone, methylisothiazolinone, and combinations thereof.

28. The method of any one of the preceding claims, wherein the cosmetic composition further comprises a moisturizing agent or a humectant.

29. The method of any one of the preceding claims, wherein the cosmetic composition is a lotion, cream, gel, serum, or emulsion.

30. The method of any one of the preceding claims, wherein the composition is formulated as a cream.

31. The method of any one of the preceding claims, wherein the cosmetic composition is a cleanser, a toner, a moisturizer, or a mask.

32. The method of any one of the preceding claims, wherein the cosmetic composition comprises 0.001% to 80% by weight of lysed adipose tissue.

33. The method of any one of the preceding claims, wherein the composition is administered daily.

34. A method of rejuvenating skin, comprising: topically administering to a subject a cosmetic composition comprising an adipose derived hormone, and a dermatological acceptable vehicle.

35. The method of any one of the preceding claims, wherein the adipose derived hormone includes leptin, adiponectin, and combinations thereof.

36. A method of preparing a fat lysate topical formulation, comprising:
a. removing adipose-containing tissue from a subject;
b. lysing the adipose-containing tissue; and
c. combining the lysed adipose-containing tissue with a topically applicable base.

37. The method of any one of the preceding claims, wherein the adipose-containing tissue is removed by a lipectomy procedure.

38. The method of any one of the preceding claims, wherein the lipectomy procedure is selected from suction assisted lipectomy (liposuction), dermal lipectomy procedures used for body contouring (such as abdominoplasty, brachioplasty, thigh-plasty, belt-lipectomy, buttock lifting), and isolated lipectomy (direct excision or suction assisted) to obtain fat for the topical formulation only.

39. The method of any one of the preceding claims further comprising filtering or purifying the lysed adipose-containing tissue before combining with the topically applicable base.

40. The method of any one of the preceding claims, wherein lysing the adipose-containing tissue eliminates any live cells.

41. The method of any one of the preceding claims, wherein 200 μg of lysed adipose-containing tissue is combined with every 1 mL of the base.

42. A method of preparing a fat lysate topical formulation, comprising:
a. obtaining adipose-containing tissue removed from a subject;
b. lysing the adipose-containing tissue; and
c. combining the lysed adipose-containing tissue with a topically applicable base.

43. The method of any one of the preceding claims, wherein lysing the adipose-containing tissue eliminates any live cells.

44. The method of any one of the preceding claims, wherein 200 μg of lysed adipose-containing tissue is combined with every 1 mL of the base.

45. The method of any one of the preceding claims, wherein adipose-containing tissue is subcutaneous fat.
